(19) United States
(12) Patent Application Publication
(10) Pub. No.: US 2025/0282816 A1
(43) Pub. Date: Sep. 11, 2025

(54) THIOSTREPTON COMPOSITIONS AND PREPARATION THEREOF

(71) Applicant: RS Oncology, LLC, Cambridge, MA (US)

(72) Inventors: Jarrett B. Duncan, Dunbarton, NH (US); George N. Naumov, Brookline, MA (US); Rodney E. Thompson, Cornelius, NC (US); Adirà Espinà Torres, Sant Cugat Del Vallés (ES); Xavier Pujol Ollé, Barcelona (ES); Lluís Sastre Sorolla, Vallirana (ES)

(21) Appl. No.: 18/691,631

(22) PCT Filed: Sep. 16, 2022
(86) PCT No.: PCT/US22/43772

§ 371 Date: Mar. 13, 2024

(30) Foreign Application Priority Data
Sep. 17, 2021 (EP) 21382839.5

(51) Int. Cl. C07K 1/30; A61K 9/08; A61K 38/16; A61K 47/14; A61K 47/20; C07K 14/36

(52) U.S. Cl. CPC C07K 1/306; A61K 9/08; A61K 38/164; A61K 47/14; A61K 47/20; C07K 14/36

(57) ABSTRACT

Disclosed are ultrapure preparations of thiostrepton, pharmaceutical composition comprising such preparations, as well as methods of preparing such preparations.

### CROSS-REFERENCE TO RELATED APPLICATIONS

This application is a National Stage of International Application No. PCT/US22/43772, filed on Sep. 16, 2022, which claims the benefit of European Patent Application No. 21382839.5 filed on Sep. 17, 2021, each of which is hereby incorporated by reference in its entirety.

### BACKGROUND

Thiostrepton is a cyclic oligopeptide antibiotic that is also known by other names such as Bryamycin, Thiactin, alaninamide, HR4S203Y18, etc. Recent studies have shown that thiostrepton also has promising anticancer activity. Current methods of making and purifying thiostrepton, however, provide material that is undesirable for human use due to the presence of impurities and excess residual solvent. There thus remains a need for high purity preparations of thiostrepton that are preferable for administration to a human subject.

### SUMMARY

In certain embodiments, the present invention provides ultrapure preparations of thiostrepton having a purity of at least about 98% (w/w), wherein the preparation comprises less than or equal to:
a. 3000 ppm methanol;
b. 600 ppm dichloromethane;
c. 60 ppm chloroform; and
d. 410 ppm acetonitrile.

In certain embodiments, the present invention also provides pharmaceutical compositions comprising the ultrapure preparation of thiostrepton disclosed herein, in combination with one or more pharmaceutically acceptable excipients or carriers.

Also provided herein are methods of treating cancer, comprising administering to a subject in need thereof any of the pharmaceutical compositions described herein.

In certain embodiments, the present invention also provides methods of purifying thiostrepton comprising the steps of:
(1) dissolving thiostrepton in a first solvent to generate a first thiostrepton solution;
(2) distilling solvent impurities from the first thiostrepton solution to generate a second thiostrepton solution;
(3) combining a second solvent with the second thiostrepton solution to precipitate thiostrepton and thus generate a first thiostrepton solid and a third solution;
(4) washing the first thiostrepton solid with a third solvent to remove impurities and thus generate a second thiostrepton solid; and
(5) drying the second thiostrepton solid to remove residual solvent;
thereby producing an ultrapure preparation of thiostrepton.

### BRIEF DESCRIPTION OF THE DRAWINGS

FIG. 1 contains a schematic of the good manufacturing practice (GMP) crystallization process.

FIG. 2 contains a schematic of the GMP residual solvent displacement process.

FIG. 3 contains a schematic of two solvent displacement processes.

FIG. 4 is a chart showing response to commercial TS (Non-GMP) and ultrapure TS (GMP) of SKOV3 cells treated for 48 hours with indicated concentrations of compounds. N=4 replicates.

FIG. 5 is a chart showing EC50 values of commercial TS (Non-GMP) and ultrapure TS (GMP) of SKOV3 cells. N=4 replicates, ****p<0.0001, Unpaired t test.

### DETAILED DESCRIPTION

In certain aspects, this disclosure provides preparations of thiostrepton. In certain aspects, the preparations are ultrapure preparations of thiostrepton. Thiostrepton is a drug substance or active pharmaceutical ingredient that can be formulated in many different ways, and some exemplary pharmaceutical preparations are set forth below.

In certain embodiments, the ultrapure preparation of thiostrepton has greater than about 98% purity; greater than about 99% purity; greater than about 99.5% purity; or greater than about 99.9% purity.

In certain embodiments, the ultrapure preparation of thiostrepton comprises less than 3000 ppm methanol; less than about 1000 ppm methanol; less than about 500 ppm methanol; less than about 300 ppm methanol; less than about 100 ppm methanol; or less than about 50 ppm methanol. In certain embodiments, the ultrapure preparation of thiostrepton comprises methanol in a concentration that is at least the lower limit of detection for methanol. In certain embodiments, the ultrapure preparation of thiostrepton comprises from about 1 ppm to 3000 ppm, or from about 1 ppm to about 1000 ppm, or from about 1 ppm to about 500 ppm, or from about 1 ppm to about 50 ppm methanol.

In certain embodiments, the ultrapure preparation of thiostrepton comprises less than 600 ppm dichloromethane; less than about 100 ppm dichloromethane; less than about 60 ppm dichloromethane; less than about 20 ppm dichloromethane; or less than about 10 ppm dichloromethane. In certain embodiments, the ultrapure preparation of thiostrepton comprises dichloromethane in a concentration that is at least the lower limit of detection for dichloromethane. In certain embodiments, the ultrapure preparation of thiostrepton comprises from about 1 ppm to 600 ppm, or from about 1 ppm to about 100 ppm, or from about 1 ppm to about 60 ppm, or from about 1 ppm to about 20 ppm, or from about 1 ppm to about 10 ppm dichloromethane.

In certain embodiments, the ultrapure preparation of thiostrepton comprises less than 60 ppm chloroform; less than about 30 ppm chloroform; less than about 10 ppm chloroform; or less than about 5 ppm chloroform. In certain embodiments, the ultrapure preparation of thiostrepton comprises chloroform in a concentration that is at least the lower limit of detection for chloroform. In certain embodiments, the ultrapure preparation of thiostrepton comprises from about 1 ppm to 60 ppm, or from about 1 ppm to about 30 ppm, or from about 1 ppm to about 10 ppm, or from about 1 ppm to about 5 ppm chloroform.

In certain embodiments, the ultrapure preparation of thiostrepton comprises less than 410 ppm acetonitrile; less than about 200 ppm acetonitrile; less than about 150 ppm acetonitrile; less than about 100 ppm acetonitrile; or less than about 50 ppm acetonitrile. In certain embodiments, the ultrapure preparation of thiostrepton comprises acetonitrile in a concentration that is at least the lower limit of detection for acetonitrile. In certain embodiments, the ultrapure preparation of thiostrepton comprises from about 1 ppm to 410 ppm, or from about 1 ppm to about 200 ppm, or from about 1 ppm to about 150 ppm, or from about 1 ppm to about 100 ppm, or from about 1 ppm to about 50 ppm acetonitrile.

In certain embodiments, the ultrapure preparation of thiostrepton has greater than 98% purity, and comprises less than 3000 ppm methanol; less than 600 ppm dichloromethane; less than 60 ppm chloroform; and less than 410 ppm acetonitrile. In certain embodiments, the ultrapure preparation of thiostrepton has greater than about 99% purity, and comprises less than about 300 ppm methanol; less than about 60 ppm dichloromethane; less than about 60 ppm chloroform; and less than about 200 ppm acetonitrile. In certain embodiments, the ultrapure preparation of thiostrepton has greater than about 99% purity, and comprises less than about 100 ppm methanol; less than about 20 ppm dichloromethane; less than about 60 ppm chloroform; and less than about 150 ppm acetonitrile.

In certain embodiments, the invention provides a pharmaceutical composition comprising any of the ultrapure preparations of thiostrepton set forth herein and one or more pharmaceutically acceptable excipients or carriers. In certain embodiments, the composition is an aqueous composition.

In certain embodiments, the composition comprises from about 0.1 to about 10 mg of ultrapure thiostrepton per mL of water. In certain embodiments, the composition comprises from about 1 to about 5 mg of ultrapure thiostrepton per mL of water. In certain embodiments, the composition comprises about 3 mg of ultrapure thiostrepton per mL of water.

In certain embodiments, the composition further comprises Vitamin E-TPGS. In certain embodiments, the composition comprises from about 0.01 to about 0.5 g of Vitamin E-TPGS per mL of water. In certain embodiments, the composition comprises from about 0.05 to about 0.1 g of Vitamin E-TPGS per mL of water. In certain embodiments, the composition comprises about 0.07 g of Vitamin E-TPGS per mL of water.

In certain embodiments, the composition further comprises dimethyl sulfoxide (DMSO). In certain embodiments, the composition comprises from about 0.005 to about 0.05 g of DMSO per mL of water. In certain embodiments, the composition comprises from about 0.01 to about 0.03 g of DMSO per mL of water. In certain embodiments, the composition comprises about 0.017 g of DMSO per mL of water.

In certain embodiments, the composition comprises about 3 mg of ultrapure thiostrepton per mL of water, about 0.07 g of Vitamin E-TPGS per mL of water and about 0.017 g of DMSO per mL of water.

In certain embodiments, the composition also includes one or more of a pharmaceutically acceptable carrier and excipient. Formulations of thiostrepton are disclosed in WO 2020/142782, the contents of which are incorporated by reference herein.

In one aspect, provided are methods of purifying thiostrepton comprising the steps of:
(1) dissolving thiostrepton in a first solvent to generate a first thiostrepton solution;
(2) distilling solvent impurities from the first thiostrepton solution to generate a second thiostrepton solution;
(3) combining a second solvent with the second thiostrepton solution to precipitate thiostrepton and thus generate a first thiostrepton solid and a third solution;
(4) washing the first thiostrepton solid with a third solvent to remove impurities and thus generate a second thiostrepton solid and a fourth solution; and
(5) drying the second thiostrepton solid to remove residual solvent;
thereby producing the ultrapure preparation of thiostrepton.

In certain embodiments, the method comprises the step (1) of combining thiostrepton and a first solvent to generate a first thiostrepton solution. In certain embodiments, the first solvent is a solvent in which thiostrepton is highly soluble (i.e., having a solubility of at least 25 mg/mL). In certain embodiments, the first solvent comprises a chlorinated solvent. In certain embodiments, the chlorinated solvent comprises chloroform. In certain embodiments, the first solvent comprises chloroform and an alcohol. In certain embodiments, the first solvent comprises from about 0.5 to about 10% (v/v) ethanol. In certain embodiments, the chlorinated solvent comprises from about 0.5 to about 5.0% (v/v) ethanol. In certain embodiments, the chloroform comprises from about 0.5 to about 1.0% (v/v) ethanol. In certain embodiments, the alcohol is ethanol. In certain embodiments, the first solvent has a pH of from about 4.0 to about 7.0. In certain embodiments, the first solvent has a pH of from about 5.0 to about 6.0. In certain embodiments, the temperature of the first solvent is from about 30° C. to about 60° C. In certain embodiments, the temperature of the first solvent is from about 40° C. to about 50° C.

In certain embodiments, the method further comprises the step of cooling the first thiostrepton solution to a temperature of from about 10° C. to about 35° C. In certain embodiments, the thiostrepton solution is cooled to a temperature of from about 15° C. to about 30° C.

In certain embodiments, the method further comprises the step (2) of distilling solvent impurities from the first thiostrepton solution to generate a second thiostrepton solution. In certain embodiments, distilling the first thiostrepton solution occurs under a reduced pressure. In certain embodiments, distilling the first thiostrepton solution at reduced pressure is performed at a temperature of from about 35° C. to about 70° C. In certain embodiments, distilling the first thiostrepton solution at reduced pressure is performed at a temperature of from about 40° C. to about 50° C. In certain embodiments, the volume of the second thiostrepton solution is at least about 30% less than the volume of the first thiostrepton solution. In certain embodiments, the volume of the second thiostrepton solution is at least about 50% less than the volume of the first thiostrepton solution. In certain embodiments, the volume of the second thiostrepton solution is at least about 70% less than the volume of the first thiostrepton solution.

In certain embodiments, the method further comprises the step of cooling the second thiostrepton solution to a temperature of from about 10° C. to about 30° C. In certain embodiments, the second thiostrepton solution is cooled to a temperature of from about 15° C. to about 25° C. In certain embodiments, the second thiostrepton solution is cooled to a temperature of at most about 25° C. In certain embodiments, the second thiostrepton solution is cooled to a temperature of at most about 20° C. In certain embodiments, the second thiostrepton solution is cooled to a temperature of at most about 15° C.

In certain embodiments, the method further comprises the step (3) of combining a second solvent with the second thiostrepton solution to precipitate thiostrepton and thus generate a first thiostrepton solid and a third solution (i.e., the mother liquor). In certain embodiments, the second solvent is an organic solvent. In certain embodiments, the organic solvent is a poor solvent for thiostrepton (i.e., an antisolvent). In certain embodiments, the second solvent is acetonitrile. In certain embodiments, the volume of the second solvent is about equal to the volume of the second thiostrepton solution. In certain embodiments, the second solvent is added to the second thiostrepton solution with stirring.

A poor solvent for thiostrepton is a solvent in which thiostrepton has solubility of less than 1 g/100 mL, 0.5 g/100 mL, or 0.1 g/100 mL.

In certain embodiments, the method comprises washing the first thiostrepton solid with one or more portions of a washing solvent that comprises a poor solvent for thiostrepton. In certain embodiments, the washing solvent comprises acetonitrile. In certain embodiments, the washing solvent comprises a mixture of acetonitrile and chloroform. In certain embodiments, the washing solvent is a 1:1 mixture acetonitrile and chloroform.

In certain embodiments, the method further comprises separating the first thiostrepton solid from the third solution and drying the first solid.

In certain embodiments, the method comprises the step (4) of washing the first thiostrepton solid with a third solvent to generate a second thiostrepton solid. In certain embodiments, washing the first thiostrepton solid with a third solvent comprises soaking the first thiostrepton solid in the third solvent. In some embodiments, soaking the first thiostrepton solid in the third solvent is performed with stirring. In some embodiments, the third solvent comprises water. In some embodiments, the third solvent comprises water and acetonitrile. In certain embodiments, the water and the acetonitrile has a volume to volume ratio of from about 20:1 to about 1:1. In certain embodiments, the water and the acetonitrile has a volume to volume ratio of from about 10:1 to about 1:1. In certain embodiments, the water and the acetonitrile has a volume to volume ratio of from about 5:1 to about 1:1. In certain embodiments, the water and the acetonitrile has a volume to volume ratio of about 10:1. In certain embodiments, the water and the acetonitrile has a volume to volume ratio of about 8:1. In certain embodiments, the water and the acetonitrile has a volume to volume ratio of about 4:1.

In certain embodiments, washing the first thiostrepton solid with a third solvent is performed for a duration of at least about 30 minutes. In certain embodiments, washing the first thiostrepton solid with a third solvent is performed for a duration of at least about 60 minutes. In certain embodiments, washing the first thiostrepton solid with a third solvent is performed for a duration of at least about 90 minutes.

In certain embodiments, the second thiostrepton solid comprises less residual solvent e.g., methanol, dichloromethane, chloroform and/or acetonitrile than the first thiostrepton solid. Without wishing to be bound by a theory, it is thought that the washing in step (4) removes and or exchanges solvent molecules that are trapped in the cake of the first thiostepton solid after it is separated from the third solution.

In certain embodiments, the method comprises the additional step of washing the second thiostrepton solid with at least one further portion of the third solvent following the step of washing the first solid with a third solvent to generate the second solid s.

In certain embodiments, the method comprises the additional step of washing the second thiostrepton solid with a fourth solvent following the step of washing the first thiostrepton solid with a third solvent to generate the second thiostrepton solid. In certain embodiments, washing the second solid with a fourth solvent comprises soaking the second solid in the fourth solvent. In certain embodiments, the fourth solvent comprises water. In certain embodiments, the fourth solvent is water.

In certain embodiments, washing the second thiostrepton solid with a fourth solvent is performed for a duration of at least about 30 minutes. In certain embodiments, washing the second thiostrepton solid with a fourth solvent is performed for a duration of at least about 60 minutes. In certain embodiments, washing the second thiostrepton solid with a fourth solvent is performed for a duration of at least about 90 minutes.

In certain embodiments, soaking the second thiostrepton solid in the fourth solvent is performed at an elevated temperature and under reduced pressure. In certain embodiments, soaking the second thiostrepton solid in the fourth solvent is performed at an elevated temperature and under reduced pressure for at least 3 hours, 6 hours, or 10 hours. In certain embodiments, the elevated temperature is from about 50 to about 60° C. In certain embodiments, the elevated temperature is from about 60 to about 70° C. In certain embodiments, the elevated temperature is from about 70 to about 80° C. In certain embodiments, the elevated temperature is at least about 50° C. In certain embodiments, the elevated temperature is at least about 60° C. In certain embodiments, the reduced pressure is vacuum.

In certain embodiments, the method further comprises separating the second thiostrepton solid from the fourth solution and step (5) drying the second thiostrepton solid to remove residual solvent. In certain embodiments, drying the second thiostrepton solid is performed under reduced pressure.

In certain embodiments, the second thiostrepton solid is analyzed for methanol, acetonitrile, dichloromethane and chloroform content. In certain embodiments, if the precipitate comprises greater than 3000 ppm methanol, 600 ppm dichloromethane, 60 ppm chloroform, and/or 410 ppm acetonitrile, the second solid is subjected to further cycles of steps (4) and (5). In certain embodiments, the number of further cycles of steps (4) and (5) is 1. In certain embodiments, the number of further cycles of steps (4) and (5) is 2. In certain embodiments, the number of further cycles of steps (4) and (5) is 3. In certain embodiments, the number of further cycles of steps (4) and (5) is 4.

In certain embodiments, the method comprises the steps of: a. dissolving crude thiostrepton in chloroform containing 0.5-1.0% (v/v) ethanol at a pH of between about 5.0-6.0 at a temperature of between 40°-50° C.; b. cooling the solution of step a. to a temperature of 15-30° C.; c. heating the cooled solution to a temperature of between 40°-50° C. and distilling under vacuum to reduce volume by at least 50%; d. cooling the distilled solution to a temperature of 15-25° C.; e. adding an equal volume of acetonitrile to the cooled solution with stirring until a precipitate forms; f. collecting and drying the precipitate; g. soaking the dried precipitate in a 4:1 mixture of H₂O:acetonitrile; h. draining any residual liquid from the precipitate; i. soaking the precipitate in H₂O; j. draining any residual liquid from the precipitate; and k. drying the precipitate; to produce the ultrapure preparation of thiostrepton.

In certain embodiments, step (1) is performed on a thiostrepton scale of greater than 10 grams. In certain embodiments, step (1) is performed on a thiostrepton scale of greater than 1 kilogram. In certain embodiments, step (1) is performed on a thiostrepton scale of greater than 10 kilograms. In certain embodiments, wherein step (1) is performed on a thiostrepton scale of from about 100 milligrams to about 100 kilograms, from about 100 milligrams to about 10 kilograms, from about 1.0 gram to about 10 kilograms, from about 1.0 gram to about 10 kilograms, from about 1.0 gram to about 1.0 kilograms, or from about 20.0 grams to about 100 grams. In certain embodiments, the invention provides an ultrapure preparation of thiostrepton prepared according to any one of the methods described herein.

In certain embodiments, the invention provides a method of treating cancer, comprising administering to a subject in need thereof any one of the pharmaceutical compositions described herein.

In certain embodiments, the residual solvents present in the ultrapure preparation of thiostrepton of the present invention may be determined according to the procedures outlined in USP <467>. USP <467> establishes, among other things, procedures for establishing exposure limits of residual solvents in pharmaceutical products.

### Pharmaceutical Compositions

In certain embodiments, the ultrapure preparation of thiostrepton of the invention, or the ultrapure preparation of thiostrepton prepared according to any one of the methods described herein, will be formulated in a pharmaceutical composition. For example, the pharmaceutical composition may comprise the ultrapure preparation of thiostrepton and a pharmaceutically acceptable carrier. As a further example, the pharmaceutical composition may comprise the ultrapure preparation of thiostrepton prepared according to any one of the methods described herein and a pharmaceutically acceptable carrier.

In certain embodiments, the pharmaceutical composition comprises less than 3000 ppm methanol; less than about 1000 ppm methanol; less than about 500 ppm methanol; less than about 300 ppm methanol; less than about 100 ppm methanol; or less than about 50 ppm methanol. In certain embodiments, the pharmaceutical composition comprises methanol in a concentration that is at least the lower limit of detection for methanol. In certain embodiments, the pharmaceutical composition comprises from about 1 ppm to 3000 ppm, or from about 1 ppm to about 1000 ppm, or from about 1 ppm to about 500 ppm, or from about 1 ppm to about 50 ppm methanol.

In certain embodiments, the pharmaceutical composition comprises less than 600 ppm dichloromethane; less than about 100 ppm dichloromethane; less than about 60 ppm dichloromethane; less than about 20 ppm dichloromethane; or less than about 10 ppm dichloromethane. In certain embodiments, the pharmaceutical composition comprises dichloromethane in a concentration that is at least the lower limit of detection for dichloromethane. In certain embodiments, the pharmaceutical composition comprises from about 1 ppm to 600 ppm, or from about 1 ppm to about 100 ppm, or from about 1 ppm to about 60 ppm, or from about 1 ppm to about 20 ppm, or from about 1 ppm to about 10 ppm dichloromethane.

In certain embodiments, the pharmaceutical composition comprises less than 60 ppm chloroform; less than about 30 ppm chloroform; less than about 10 ppm chloroform; or less than about 5 ppm chloroform. In certain embodiments, the pharmaceutical composition comprises chloroform in a concentration that is at least the lower limit of detection for chloroform. In certain embodiments, the pharmaceutical composition comprises from about 1 ppm to 60 ppm, or from about 1 ppm to about 30 ppm, or from about 1 ppm to about 10 ppm, or from about 1 ppm to about 5 ppm chloroform.

In certain embodiments, the pharmaceutical composition comprises less than 410 ppm acetonitrile; less than about 200 ppm acetonitrile; less than about 150 ppm acetonitrile; less than about 100 ppm acetonitrile; or less than about 50 ppm acetonitrile. In certain embodiments, the pharmaceutical composition comprises acetonitrile in a concentration that is at least the lower limit of detection for acetonitrile. In certain embodiments, the pharmaceutical composition comprises from about 1 ppm to 410 ppm, or from about 1 ppm to about 200 ppm, or from about 1 ppm to about 150 ppm, or from about 1 ppm to about 100 ppm, or from about 1 ppm to about 50 ppm acetonitrile.

In certain embodiments, the compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, an ultrapure preparation of thiostrepton and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or organic esters.

A pharmaceutically acceptable carrier can contain physiologically acceptable agents that act, for example, to stabilize, increase solubility or to increase the absorption of a compound such as thiostrepton. Such physiologically acceptable agents include, for example, carbohydrates, such as glucose, sucrose or dextrans, antioxidants, such as ascorbic acid or glutathione, chelating agents, low molecular weight proteins or other stabilizers or excipients.

Further examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Methods of preparing these formulations or compositions include the step of bringing into association an active compound, such as thiostrepton, with the carrier and, optionally, one or more accessory ingredients. In general, the formulations are prepared by uniformly and intimately bringing into association an active with one or more liquid carriers.

Suspensions, in addition to the active compound(s), may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, the route of administration, the time of administration, the rate of clearance or excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of active agent being administered with thiostrepton. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods described herein will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments, the active compound may be administered two or three times daily. In certain embodiments, the active compound will be administered once daily.

The patient receiving this treatment is any animal in need, including primates, in particular humans, and other mammals such as equines, cattle, swine and sheep; and poultry and pets in general.

In certain embodiments, ultrapure preparations of thiostrepton may be used alone or conjointly administered with another type of active agent. As used herein, the phrase “conjoint administration” refers to any form of administration of two or more different active compounds such that the second compound is administered while the previously administered active compound is still effective in the body (e.g., the two compounds are simultaneously effective in the patient, which may include synergistic effects of the two compounds). For example, the different active compounds can be administered either in the same formulation or in a separate formulation, either concomitantly or sequentially. In certain embodiments, the different active compounds can be administered within one hour, 12 hours, 24 hours, 36 hours, 48 hours, 72 hours, or a week of one another. Thus, an individual who receives such treatment can benefit from a combined effect of different active compounds.

In certain embodiments, conjoint administration of ultrapure preparations of thiostrepton with one or more additional active agent(s) (e.g., one or more additional chemotherapeutic agent(s)) provides improved efficacy relative to each individual administration of the ultrapure preparation of thiostrepton or the one or more additional active agent(s). In certain such embodiments, the conjoint administration provides an additive effect, wherein an additive effect refers to the sum of each of the effects of individual administration of the ultrapure preparation of thiostrepton and the one or more additional active agent(s).

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic (i.e., it is intended to diminish, ameliorate, or stabilize the existing unwanted condition or side effects thereof). Treating may also encompass eliminating the unwanted condition or side effect. As used herein, treating a disease, disorder, or condition includes treating complication(s) of the disease, disorder, or condition, such as by treating the underlying pathophysiology specific to the complication(s) of the disease, disorder, or condition. The subject to whom the active agent is administered may be asymptomatic or symptomatic.

In certain embodiments, a composition comprising the ultrapure preparation of thiostrepton and Vitamin E-TPGS is administered to cells and/or a subject using a catheter to infuse the composition into a body cavity of a subject and/or to wash a body cavity of a subject with the composition. Examples of catheters that may be used in certain embodiments of the invention include, but are not limited to, an intra-pleural catheter and an intra-peritoneal catheter. In a non-limiting example, an intra-pleural catheter is used to administer one or more doses of a composition of the invention comprising the ultrapure preparation of thiostrepton and Vitamin E-TPGS into the plural cavity a subject with a plural effusion. In another non-limiting example, a subject with ovarian cancer may be administered a composition of the invention comprising the ultrapure preparation of thiostrepton and Vitamin E-TPGS to the peritoneal cavity using an intra-peritoneal catheter.

### Definitions

“NLT” is an art-recognized term meaning “Not Less Than”.

“Soaking” as used herein refers to immersing a solid material in a liquid.

“TS” as used herein refers to thiostrepton.

“Ultrapure” as used herein refers to a substance having a purity of at least about 98% (w/w) and less than or equal to: 3000 ppm methanol, 600 ppm dichloromethane, 60 ppm chloroform and/or 410 ppm acetonitrile i.e., in compliance with ICH (International Conference on Harmonisation of Technical Requirements for Registration of Pharmaceuticals for Human Use) requirements for Class 2 solvents.

“Vitamin E-TPGS” is an art-recognized term and refers to D-α-tocopheryl polyethylene glycol succinate. Vitamin E-TPGS is also known as D-α-tocopheryl polyethylene glycol 1000 succinate. The terms “Vitamin E-TPGS”, “VitE” and “VitE-TPGS” are used interchangeably herein.

“v/v” is an art-recognized term and refers the proportion of a particular substance within a mixture, as measured by volume.

“w/w” is an art-recognized term and refers the proportion of a particular substance within a mixture, as measured by weight.

### PREFERRED EMBODIMENTS

1. An ultrapure preparation of thiostrepton having a purity of at least about 98% (w/w); wherein the preparation comprises less than or equal to:
a. 3000 ppm methanol;
b. 600 ppm dichloromethane;
c. 60 ppm chloroform; and
d. 410 ppm acetonitrile.

2. The ultrapure preparation of thiostrepton of embodiment 1, wherein the purity of thiostrepton is at least about 99% (w/w).

3. The ultrapure preparation of thiostrepton of embodiment 1 or 2, wherein the preparation comprises less than or equal to 300 ppm methanol.

4. The ultrapure preparation of thiostrepton of embodiment 3, wherein the preparation comprises less than or equal to 100 ppm methanol.

5. The ultrapure preparation of thiostrepton of any one of embodiments 1-4, wherein the preparation comprises less than or equal to 60 ppm dichloromethane.

6. The ultrapure preparation of thiostrepton of embodiment 5, wherein the preparation comprises less than or equal to 20 ppm dichloromethane.

7. The ultrapure preparation of thiostrepton of any one of embodiments 1-6, wherein the preparation comprises less than or equal to 200 ppm acetonitrile.

8. The ultrapure preparation of thiostrepton of any one of embodiment 7, wherein the preparation comprises less than or equal to 150 ppm acetonitrile.

9. A pharmaceutical composition, comprising the ultrapure preparation of thiostrepton of any one of embodiments 1-8; and one or more pharmaceutically acceptable excipients or carriers.

10. The pharmaceutical composition of embodiment 9, wherein the pharmaceutical composition is an aqueous composition.

11. The pharmaceutical composition of embodiment 10, wherein the pharmaceutical composition comprises from about 1 to about 5 mg of ultrapure thiostrepton per mL of water.

12. The pharmaceutical composition of embodiment 11, wherein the pharmaceutical composition comprises about 3 mg of ultrapure thiostrepton per mL of water.

13. The pharmaceutical composition of any one of embodiments 10-12, further comprising Vitamin E-TPGS.

14. The pharmaceutical composition of embodiment 13, wherein the pharmaceutical composition comprises from about 0.05 to about 0.1 g of Vitamin E-TPGS per mL of water.

15. The pharmaceutical composition of any one of embodiments 10-12, further comprising dimethyl sulfoxide (DMSO).

16. The pharmaceutical composition of embodiment 15, wherein the pharmaceutical composition comprises from about 0.01 to about 0.03 g of DMSO per mL of water.

17. A method of purifying thiostrepton comprising the steps of:
(1) dissolving thiostrepton in a first solvent to generate a first thiostrepton solution;
(2) distilling solvent impurities from the first thiostrepton solution to generate a second thiostrepton solution;
(3) combining a second solvent with the second thiostrepton solution to precipitate thiostrepton and thus generate a first thiostrepton solid and a third solution;
(4) washing the first thiostrepton solid with a third solvent to remove impurities and thus generate a second thiostrepton solid; and
(5) drying the second thiostrepton solid to remove residual solvent;
thereby producing an ultrapure preparation of thiostrepton.

18. The method of embodiment 16 or 17, wherein the first solvent comprises chloroform.

19. The method of embodiment 18, wherein the first solvent comprises from about 0.5 to about 1.0% (v/v) ethanol.

20. The method of any one of embodiments 16-19, wherein the first solvent has a pH of from about 5 to about 6.

21. The method of any one of embodiments 16-20, wherein the first solvent is at a temperature of from about 40° C. to about 50° C.

22. The method of any one of embodiments 16-21, wherein step (1) further comprises cooling the first thiostrepton solution to a temperature of from about 15 to about 30° C.

23. The method of any one of embodiments 16-22, wherein step (2) comprises distilling the first thiostrepton solution at reduced pressure.

24. The method of any one of embodiments 16-23, wherein distilling the first thiostrepton solution is performed at a temperature of from about 40° C. to about 50° C.

25. The method of any one of embodiments 16-24, wherein the volume of the second thiostrepton solution is at least about 50% less than the volume of the first thiostrepton solution.

26. The method of any one of embodiments 16-25, wherein step (2) further comprises cooling the second thiostrepton solution to a temperature of from about 15° C. to about 25° C.

27. The method of any one of embodiments 16-26, wherein the second solvent comprises acetonitrile.

28. The method of any one of embodiments 16-27, wherein the volume of the second solvent is about equal to the volume of the second thiostrepton solution.

29. The method of any one of embodiments 16-28, wherein the second solvent is added to the second thiostrepton solution with stirring.

30. The method of any one of embodiments 16-29, wherein step (3) further comprises separating the first thiostrepton solid from the third solution and drying the first thiostrepton solid.

31. The method of any one of embodiments 16-30, wherein the third solvent comprises water.

32. The method of any one of embodiments 16-31, wherein the third solvent is a mixture of water and acetonitrile.

33. The method of any one of embodiments 16-32, wherein the third solvent is about a 4:1 v/v mixture of water and acetonitrile.

34. The method of any one of embodiments 16-33, wherein washing the first thiostrepton solid with a third solvent comprises soaking the first thiostrepton solid with the third solvent.

35. The method of embodiment 34, wherein the soaking is performed for a duration of at least about 30 minutes.

36. The method of any one of embodiments 16-35, wherein the method comprises the additional step of washing the second thiostrepton solid with at least one further portion of the third solvent following the step of washing the first solid with a third solvent to generate the second solid.

37. The method of any one of embodiments 16-36, wherein the method comprises the additional step of washing the second thiostrepton solid with a fourth solvent following the step of washing the first thiostrepton solid with a third solvent to generate the second thiostrepton solid.

38. The method of any one of embodiments 16-37, wherein the fourth solvent comprises water.

39. The method of any one of embodiments 16-38, wherein the washing comprises soaking the second thiostrepton solid with the fourth solvent.

40. The method of embodiment 39, wherein the soaking is performed for a duration of at least about 30 minutes.

41. The method of embodiment 39 or 40, wherein the soaking is performed at a temperature of from about 50 to about 60° C. under vacuum for at least 10 hours.

42. The method of any one of embodiments 16-41, wherein the second thiostrepton solid is analyzed for methanol, acetonitrile, dichloromethane and/or chloroform content.

43. The method of embodiment 42, wherein if the second thiostrepton solid comprises greater than 3000 ppm methanol, 600 ppm dichloromethane, 60 ppm chloroform, and/or 410 ppm acetonitrile, the second thiostrepton solid is subjected to further cycles of steps (4) and (5).

44. The method of any one of embodiments 16-43, wherein step (a) is performed on a thiostrepton scale of from about 100 milligrams to about 100 kilograms.

45. The method of embodiment 44, wherein the step (a) is performed on a thiostrepton scale of from about 1.0 gram to about 10 kilograms.

46. The method of embodiment 45, wherein the step (a) is performed on a thiostrepton scale of from about 20.0 grams to about 1.0 kilograms.

47. An ultrapure preparation of thiostrepton prepared according to the method of any one of embodiments 17-46.

48. A method of treating cancer, comprising administering to a subject in need thereof a pharmaceutical composition of embodiments 9-16.

### EXAMPLES

In order that the invention described herein may be more fully understood, the following examples are set forth. The examples described in this application are offered to illustrate the compounds, compositions, materials, device, and methods provided herein and are not to be construed in any way as limiting their scope.

### Example 1: Manufacture of Ultrapure Preparation of Thiostrepton

Materials and Methods. To demonstrate identity and potency, both API thiostrepton, United States Pharmacopeia and purified thiostrepton Drug Substance (DS) were compared to a known USP standard using the assays specified in the USP monograph for thiostrepton:
1. Identification by IR spectrum, USP <197K>
2. Antibiotics-Microbial assay, USP <81>

In addition, head space gas chromatography assays, described in USP <467>, were utilized to measure organic impurities, inorganic impurities, and residual solvents to assure the purity of the purified thiostrepton DS after residual solvents were reduced to the ICH recommended limits for four Class 2 solvents by the DS manufacturing process. As outlined in USP <467>, the gas chromatograph was equipped with a flame-ionization detector. The second portion of the DS manufacturing process contains in-process controls to ensure these Class 2 solvent limits are met; therefore, no re-processing of the DS is allowed.

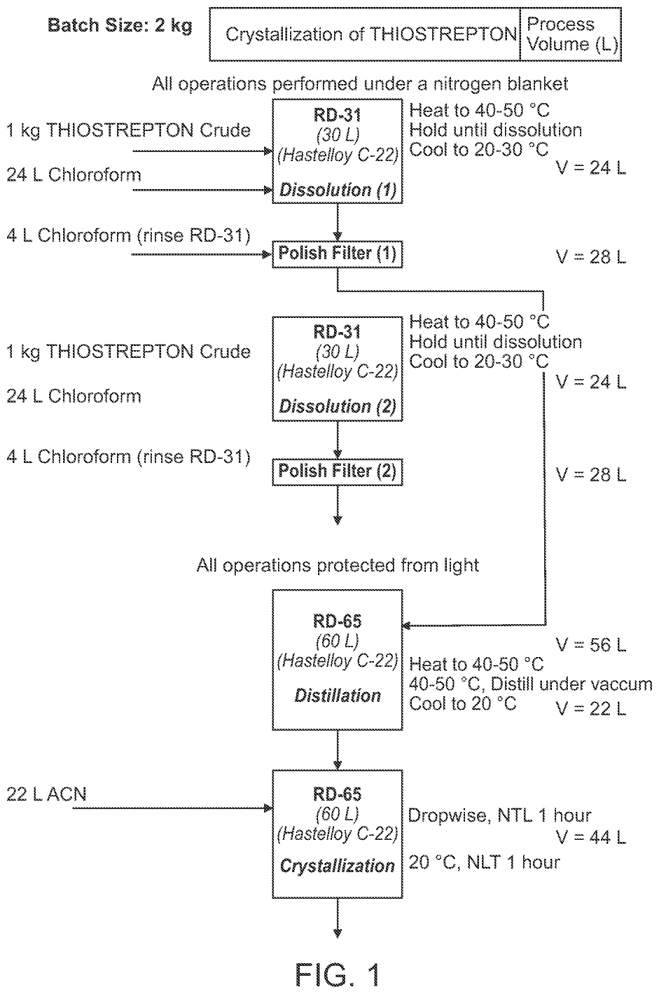

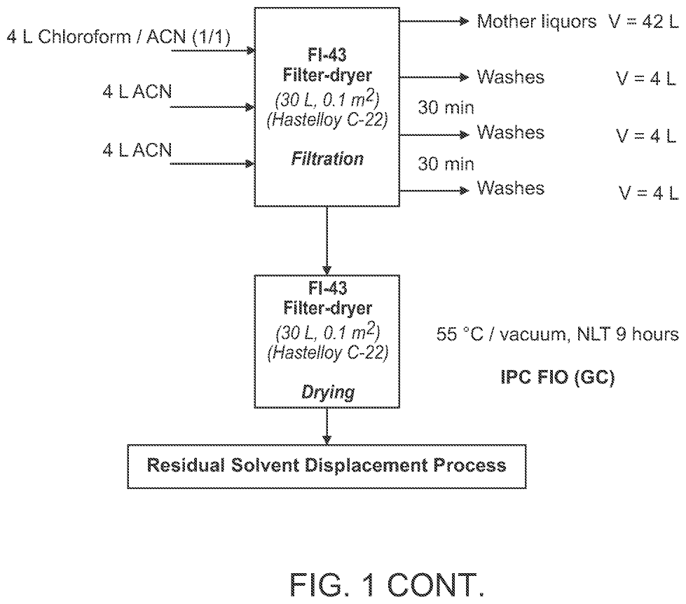

The manufacturing process for cGMP grade thiostrepton drug substance (GMP TS DS) involved a crystallization process (CP) (“A” below) followed by a residual solvent displacement process (RSDP) (“B” below). Process flow diagrams for each portion of the 2 kg scale cGMP manufacturing process (GMP DS Lot 1) are shown in FIGS. 1 and 2; comparison with the 140 g scale non-GMP.

### A. Crystallization Process (CP) (FIG. 1)

The GMP CP is presented in FIG. 1. A description of the process follows.

The chloroform used had a pH of 5-6 and contained 0.5-1.0% ethanol as a stabilizer.

The 2 kgs of thiostrepton crude API was divided into two 1-kg portions. For each portion:
1. 24 L of chloroform was charged to the 30-L Hastelloy C-22 reactor at room temperature through a 3-μm cartridge filter.
2. 1 kg of thiostrepton API was added.
3. The reactor was heated to 45° C. (40-50° C.) under stirring and held until the thiostrepton was completely dissolved yielding a clear solution.
4. The vessel was cooled to 25° C. (20-30° C.) under stirring.
5. The dissolution was transferred to the 60-L Hastelloy C-22 reactor filtering through a 3-μm cartridge polish filter.
6. The 30-L reactor, the transfer line and filter were rinsed with 4 L of chloroform and these 4 L were collected in the 60-L reactor.

The 28 L from the first portion were held at 25° C. (20-30° C.) and the 28 L from the second portion were added to the 60-L reactor and mixed with the first portion. The process continued:
1. The reactor was heated to 45° C. (40-50° C.) and the solvent distilled under vacuum under stirring.
2. When the volume was reduced to approximately 22 L, the reactor was cooled to 20° C. (15-25° C.) in approx. 30 min under stirring.
3. 22 L of acetonitrile was added over 1 hour under stirring while the temperature was maintained at 20° C. (15-25° C.).
4. The suspension was stirred for NLT 1 hour at 20° C. (18-22° C.).
5. Suspension was charged to a Hastelloy C-22 filter-dryer at 20° C. (18-22° C.).
6. Mother liquors were removed from the filter-dryer.
7. Cake was washed with a mixture of 2 L of chloroform and 2 L of acetonitrile (20° C.), the cake was removed with a scoop and drained.
8. Cake was washed with 4 L of acetonitrile (20° C.), the cake was removed with a scoop, soaked for 30 min and drained. This process step was done twice.
9. Solid was dried under vacuum at a jacket temperature of 55° C. for NLT 9 hours.
10. Filter-dryer was cooled.
11. In-process control performed (Checked the residual solvents by headspace gas chromatography-flame ionization detection (HSGC-FID)).

### B. Residual Solvent Displacement Process (RSDP) (FIG. 2)

The GMP RSDP is presented in FIG. 2. A description of the process is presented in this section. The final product from the Crystallization Process remained in the filter-dryer with 0.1 square meters of filter area. Processing continued:
1. A mixture of 6.4 L of process water and 1.6 L of acetonitrile (25° C.) was loaded, cake was removed with a scoop, soaked for 30 min and drained. This process step was done 4 times.
2. 8 L of process water (25° C.) was loaded, cake was removed with a scoop, soaked for 30 min and drained. This process step was done twice.
3. Cake was drained for at least 2 h and the solid was dried under vacuum at a jacket temperature of 60° C. for NLT 24 hours.
4. In-process control-Checked the residual solvents by headspace gas chromatography-flame ionization detection (HSGC-FID):
   a. Methanol≤3000 ppm
   b. Acetonitrile≤410 ppm
   c. Dichloromethane≤600 ppm
   d. Chloroform≤60 ppm
   e. If the IPC controls were not met, the drying time was extended another 20 hours to determine if the IPCs could be achieved. If the extended drying time was not sufficient, repeated the RSDP.
5. In-process control-Checked the loss on drying. If IPC did not meet the limit of ≤5.0%, extended the drying time and repeated the IPC until it passed.
6. Thiostrepton isolation: Discharged the solid thiostrepton in a double PE bag inside a second PE bag containing a bag of silica inside a thermo-sealed aluminium bag and inside an aluminium drum. Stored frozen at −20° C. and controlled humidity.

The resultant thiostrepton from this process is characterized below:

| Test | Specifications | | | Results | |
|---|---|---|---|---|---|
| Appearance | White to pale yellow powder. | | | White powder | |
| Identification | IR Spectrum (A TR): The infrared | | | Conforms | |
| | spectrum of Thiostrepton | | | | |
| | corresponds to that of the | | | | |
| | Thiostrepton reference standard | | | | |
| Chromatographic | >98.0% | | | 99.3% | |
| Purity (HPLC) | | | | | |
| Water content | Determine and Report | | | 3.8% | |
| Melting Point | 245° C.-255° C. | | | 245° | C. |
| Residue on ignition | ≤1.0% | | | 0.2% | |
| Loss on drying | ≤5% | | | 2% | |
| Residual Solvents | Methanol | ≤3000 | ppm | 54 | ppm |
| (HS-GC) | Acetonitrile | ≤410 | ppm | 140 | ppm |
| | Dichloromethane | ≤600 | ppm | <18 | ppm |
| | Chloroform | ≤60 | ppm | <60 | ppm |

| | | | | |
|---|---|---|---|---|
| Elemental | Cadmium (Cd) | ≤0.2 | μg/g | Complies |
| Impurities | Lead (Pb) | ≤0.5 | μg/g | Complies |
| (ICP-MS) | Arsenic (As) | ≤1.5 | μg/g | Complies |
| | Mercury (Hg) | ≤0.3 | μg/g | Complies |
| | Cobalt (Co) | ≤0.5 | μg/g | Complies |
| | Vanadium (V) | ≤1 | μg/g | Complies |
| | Nickel (Ni) | ≤2 | μg/g | Complies |

| | | | |
|---|---|---|---|
| Endotoxins | ≤0.5 EU/mg | <0.5 | EU/mg |
| Bioburden | Total Aerobic Microbial Count | <10 | CFU/g |
| | (TAMC) | | |
| | Total Yeasts and Moulds Count | | |
| | (TYMC) ≤100 CFU/g | | |
| Antibiotics- | ≥900 IU/mg | 988 | IU/mg |
| Microbial | | | |

assay

### Example 2: Further Steps to Reduce Residual Solvent Levels

The table below shows the in-process results for the process described above in Example 1.

After Solvent displacement 1 (one iteration of the Residual Solvent Displacement Process), chloroform did not reach <60 ppm after 45 hours of drying time. As a result, solvent displacement 2 (a second iteration of the Residual Solvent Displacement Process) was performed. Though chloroform reached <60 ppm, acetonitrile was above 300 ppm after 24 hours of drying time. After 31 hours of drying time, acetonitrile fell below 300 10 ppm and the process is declared finished.

Note that the in-process targets and the measured in-process values are not identical to the final product test results; for example, acetonitrile was at 220 ppm in the final in-process measurement but was found to be 140 ppm for final product testing in Example 1.

| | Drying | | | | | Loss |
|---|---|---|---|---|---|---|
| | time | Residual solvents (ppm) | | | | on |
| Process Step | (h) | Methanol | Acetonitrile | CH₂Cl₂ | Chloroform | Drying |
| In-process | — | ≤2500 | ≤300 | ≤500 | ≤50 | ≤5.0% |
| specifications | | | | | | |
| End of | — | 5808 | 86134 | ND | 16159 | — |
| crystallization | | | | | | |
| process | | | | | | |
| Solvent | 12 | 2137 | 407 | ND | 169 | 20.4% |
| displacement | | | | | | |
| 1 | | | | | | |
| Solvent | 25 | 1005 | 120 | ND | 106 | 2.1% |
| displacement | | | | | | |
| 1 | | | | | | |
| Solvent | 25 | 1476 | 140 | <18 | 140 | — |
| displacement | | | | | | |
| 1 | | | | | | |
| Solvent | 24 | 610 | 309 | ND | 11 | 4.9% |
| displacement | | | | | | |
| 2 | | | | | | |
| Solvent | 31 | 1190 | 220 | 20 | 34 | 4.4% |
| displacement | | | | | | |
| 2 | | | | | | |

### Example 3: Further Crystallization Protocols

A number of alternative crystallization protocols were explored, although many were unsatisfactory because of poor yield, purity and/or residual solvent levels.

TABLE 1
Crystallization protocols

| | | Yield | Purity | | Residual |
|---|---|---|---|---|---|
| Batch | Main goal | (%) | (%) | Description/Results | Solvent |
| L | Recrystallization | 73.2 | 98.2 | 150 volumes of THF | HSGC-FID |
| | in THF (R&D | | | (R&D) at reflux | Results |
| | quality) using | | | needed to dissolve | (ppm): |
| | water as | | | commercial TS. | MeOH: |
| | antisolvent. | | | Distil to 35 volumes. | 8917, DCM: |
| | | | | Polish filtration. | 8, |
| | | | | Seed with | Chloroform: |
| | | | | commercial TS at 25 | ND, THF: |
| | | | | ° C. Addition of 35 | 3316. |
| | | | | volumes of water as | Water (KF): |
| | | | | antisolvent at 25° C. | 1.96%. |
| | | | | Cool to 5° C. Filter | |
| | | | | at 5° C. | |
| M | Dissolution in | — | — | Dissolution used in | |
| | THF (GC | | | Batch N. | |
| | quality) at r.t. | | | 59 volumes of THF | |
| | Pool with Batch | | | (GC) at r.t. needed to | |
| | L. | | | dissolve commercial | |
| | | | | TS. | |
| N | Dissolution in | 78.8 | 98.1 | 56 volumes of THF | HSGC-FID |
| | THF (GC | | | (GC) at reflux | Results |
| | quality) at | | | needed to dissolve | (ppm): |
| | reflux. Pool | | | commercial TS. Pool | MeOH: |
| | with Batch M. | | | with TS-011. Distil | 5174, DCM: |
| | Recrystallization | | | to 35 volumes. | ND, |
| | in THF (GC | | | Polish filtration. | MTBE: 3794, |
| | quality) using | | | Seed with | Chloroform: |
| | MTBE as | | | commercial TS. | ND, THF: |
| | antisolvent. | | | Addition of 35 | 16667. |
| | | | | volumes of MTBE as | Water (KF): |
| | | | | antisolvent at 25° C. | 1.68%. |
| | | | | Filter at 20° C. | |
| O | Recrystallization | 81.7 | 97.8 | 35 volumes needed | HSGC-FID |
| | in DCM:EtOH | | | to dissolve | Results |
| | 4:1 as solvent | | | commercial TS at r.t. | (ppm): |
| | and MTBE as | | | Polish filtration. | MeOH: |
| | antisolvent | | | Seed with | 1218, EtOH: |
| | | | | commercial TS. Seed | 14219, |
| | | | | dissolved**. | DCM: 8275, |
| | | | | Addition of 35 vol of | MTBE: |
| | | | | MTBE as antisolvent | 13037, |
| | | | | at 25 C. Filter at | Chloroform: |
| | | | | 25° C. | ND. |
| | | | | | Water (KF): |
| | | | | | 1.78%. |
| P | Recrystallization | 92.1 | 96.4 | 35 volumes needed | HSGC-FID |
| | in DCM:EtOH | | | to dissolve | Results |
| | 4:1 as solvent | | | commercial TS at r.t. | (ppm): |
| | and n-heptane as | | | Polish filtration. | MeOH: |
| | antisolvent | | | Seed with | 1406, EtOH: |
| | | | | commercial TS. Seed | 13188, |
| | | | | dissolved″. Addition | DCM: 8176, |
| | | | | of 35 vol of n- | MTBE: |
| | | | | heptane as | 12939, |
| | | | | antisolvent. Filter at | Chloroform: |
| | | | | 25° C. | ND. |
| | | | | | Water (KF): |
| | | | | | 1.89%. |
| Q | Recrystallization | 83.5 | 98.1 | 35 volumes needed | HSGC-FID |
| | in DCM:EtOH | | | to dissolve | Results |
| | 4:1 as solvent | | | commercial TS at rt. | (ppm): |
| | and ACN as | | | Polish filtration. | MeOH: |
| | antisolvent. | | | Seed with | 1378, EtOH: |
| | | | | commercial TS. Seed | 12922, DUI: |
| | | | | dissolved″. Addition | 8179, |
| | | | | of 35 vol of ACN as | MTBE: |
| | | | | antisolvent. Filter at | 13529, |
| | | | | 25° C. | Chloroform: |
| | | | | | ND. |
| | | | | | Water (KF): |
| | | | | | 1.76°A). |
| R | Recrystallization | 84.7 | 97.4 | 2.7 volumes needed | HSGC-FID |
| | in DMSO as | | | to dissolve | Results |
| | solvent and | | | commercial TS at r.t. | (ppm): |
| | Water as | | | Rotavaporate the | MeOH: 239, |
| | antisolvent | | | dissolution at 45° C. | DCM: 2, |
| | | | | for 45 min to | Chloroform: |
| | | | | eliminate residual | 9, DMSO: |
| | | | | solvents***. | 7932. |
| | | | | Addition of 1 vol of | Water (KF): |
| | | | | water as antisolvent | 3.45%. |
| | | | | at 25° C. Filter at | |
| | | | | 25° C. | |
| S | Recrystallization | 78.0 | 97.2 | 3 volumes needed to | HSGC-FID |
| | in DMSO as | | | dissolve commercial | Results |
| | solvent and | | | TS at rt. | (ppm): |
| | EtOH as | | | Rotavaporate the | MeOH: 111, |
| | antisolvent. | | | dissolution at 45° C. | EtOH: 7684, |
| | | | | for 45 min to | DCM: 1. |
| | | | | eliminate residual | Chloroform: |
| | | | | solvents. Addition of | ND, DMSO: |
| | | | | 12 vol of EtOH as | 78717. |
| | | | | antisolvent at 25° C. | Water (KF): |
| | | | | Filter at 25° C. | 4.55%. |
| T | Recrystallization | 91.0 | 97.8 | 23 volumes of CHCl₃ | |
| | in Chloroform | | | at 45° C. needed to | |
| | (stabilized with | | | dissolve commercial | |
| | 0.5-1% EtOH) | | | TS. Cool to 2° C. | |
| | as solvent and | | | Seed dissolved. | |
| | MeOH as | | | Distillation to 12 | |
| | antisolvent. | | | Volumes. Cool to 2° C., | |
| | | | | opalescence. | |
| | | | | Addition of 300 mL | |
| | | | | of MeOH at 2° C. | |
| | | | | Filter at 2° C. | |
| | | | | **** | |
| U | Recrystallization | 88.6 | 98.1 | Dissolution of | |
| | in Chloroform | | | commercial TS in 23 | |
| | (stabilized with | | | vol Chloroform at 45° C. | |
| | 0.5-1% EtOH) | | | Distillation under | |
| | as solvent and | | | vacuum until 12 vol. | |
| | acetone as | | | Cool to 25° C. | |
| | antisolvent. | | | Addition of 12 vol of | |
| | | | | acetone. Filtration at | |
| | | | | 25° C. | |
| | | | | **** | |
| V | Recrystallization | 83.0 | 98.8 | Dissolution of | |
| | in Chloroform | | | commercial TS in 23 | |
| | (stabilized with | | | vol Chloroform at 45° C. | |
| | 0.5-1% EtOH) | | | Distillation under | |
| | as solvent and | | | vacuum until 12 vol. | |
| | ACN as | | | Cool to 25° C. | |
| | antisolvent. | | | Addition of 12 vol of | |
| | | | | ACN. Filtration at | |
| | | | | 25° C. | |
| | | | | **** | |
| W | Recrystallization | 27.4 | 98.3 | 63.5 volumes of THF | HSGC-FID |
| | of 5.8 g of | | | (GC) at 45° C. | Results |
| | commercial TS | | | necessary to dissolve | (ppm): |
| | in THF (GC | | | commercial TS. | MeOH: 440, |
| | quality) as | | | Distil to 28 volumes. | EtOH: 22, |
| | solvent and | | | Polish filtration. | DCM: ND, |
| | water as | | | Addition of 8.2 | Chloroform: |
| | antisolvent. | | | volumes of water as | 3, THF: 552, |
| | Suspension of | | | antisolvent at 45° C. | DMSO: |
| | the wet solid in | | | Cool to 20° C. Filter | 1352. DMSO |
| | hot water after | | | at 20° C. Suspension | contamination. |
| | filtering, to | | | of the wet solid at | |
| | eliminate THF. | | | 40° C. for 1 h in 40 | |
| | | | | vol of water. Cool to | |
| | | | | 25° C. | |
| X | Recrystallization | 68.0 | 97.5 | Dissolution of | HSGC-FID |
| | of 30 g of | | | commercial TS in 42 | Results |
| | commercial TS | | | vol Chloroform not | (ppm): |
| | in Chloroform | | | accomplished. | MeOH: 177, |
| | stabilized with | | | Addition of 5% v/v | EtOH: 282, |
| | amylene as | | | EtOH at 50° C. | ACN: 38796, |
| | solvent and | | | Brown solid formed. | DCM: 3, |
| | ACN as | | | Polish filtration. | Chloroform: |
| | antisolvent. | | | Brown solid | 1223. |
| | | | | insoluble discarded. | Water (KF): |
| | | | | Distillation under | 1.83°A. |
| | | | | vacuum until 11 vol | |
| | | | | at 45° C. jacket temp. | |
| | | | | Cool to 25° C. | |
| | | | | Addition of 11 vol | |
| | | | | ACN; not enough | |
| | | | | solid is visually | |
| | | | | observed. Addition | |
| | | | | of total 17.5 vol of | |
| | | | | ACN. Filtration at | |
| | | | | 20° C. | |
| H | Recrystallization | 85.0 | 98.4 | Dissolution of | HSGC-FID |
| | of 15 g of | | | commercial TS in 23 | Results |
| | commercial TS | | | vol Chloroform at 45° C. | (ppm): |
| | in Chloroform | | | Polish filtration. | MeOH: 82, |
| | stabilized with | | | No solid formed. | EtOH: 107, |
| | EtOH as solvent | | | Distillation under | ACN: 30733, |
| | and ACN as | | | vacuum until 11 vol | DCM: ND, |
| | antisolvent at 15 | | | at 45° C. jacket temp. | Chloroform: |
| | g scale. | | | Cool to 20° C. | 7020. |
| | | | | Addition of 11 vol | Water (KF): |
| | | | | ACN in 30 min. | 1.37%. |
| | | | | Filtration at 20° C. | |
| | | | | First wash with 2 vol | |
| | | | | of CHCl₃/ACN 1/1. | |
| | | | | Two additional | |
| | | | | washings with 2 vol | |
| | | | | of ACN. | |

Chloroform stabilized with 0.5-1.0% EtOH was identified as a preferred solvent with which MeOH, EtOH, and acetonitrile as anti-solvent yielded high purity results.

Tests of chloroform solvent and acetonitrile as antisolvent and tetrahydrofuran as solvent and water as antisolvent gave particularly high purity results and were run on larger scale in jacketed reactors. The recrystallization in chloroform as solvent and acetonitrile as antisolvent was repeated at 15 g scale with chloroform stabilized with 0.5-1% EtOH (pH: 5), and resulted in a white colored solid with 85% yield and a chromatographic purity of 98.4%.

### Example 4: Residual Solvent Displacement Process

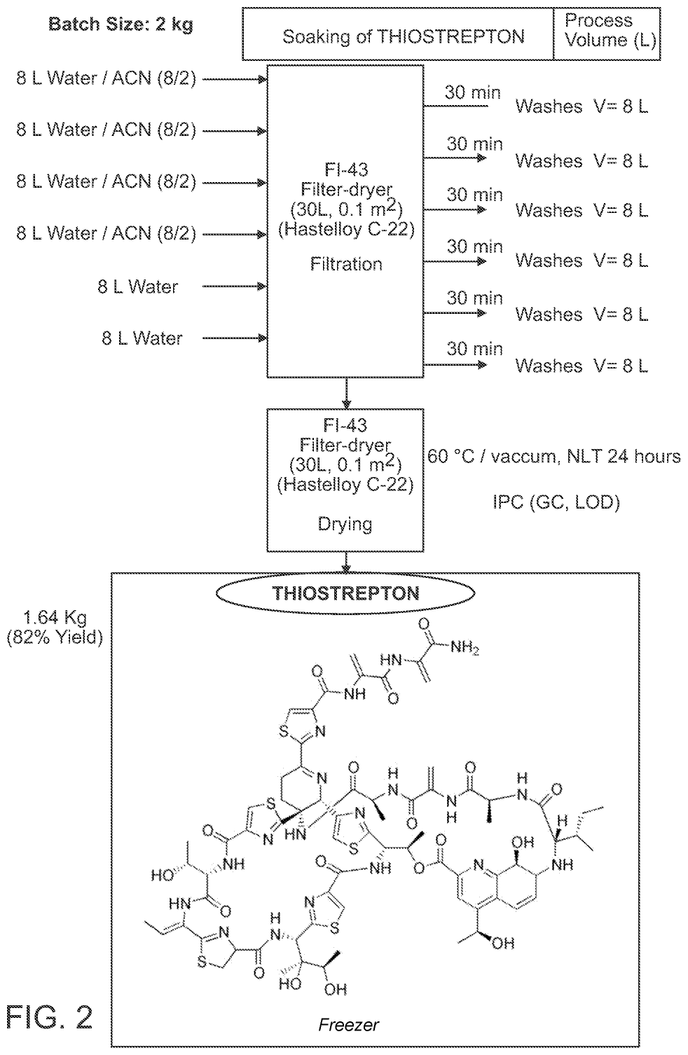

Two solvent displacement processes were developed for curing thiostrepton in order to meet residual solvent specifications in the final product: hot slurry and soaking, each one with three solvent systems (water alone, acetonitrile alone and a mixture of water/acetonitrile 2/8). The two systems that involved only water were not able to achieve ICH levels for the residual solvents and had bad operability due to the low wettability of thiostrepton because of its hydrophobicity as shown in FIG. 3.

Results for the solvent displacement processes are shown below:

| | | Starting | Scale | Residual Solvents (ppm) | | | |
|---|---|---|---|---|---|---|---|
| Batch | Conditions | Material | (g) | MeOH | DCM | ACN | CHCI₃ |
| A | Crystalization | Commercial | 30 | 177 | 3 | 38796 | 1223 |
| | CHCl₃/ACN | TS | | | | | |
| B | Water soaking | A | 8.55 | 60 | ND | 26 | 216 |
| C | Hot water slurry | | 10.63 | 86 | ND | 12 | 281 |
| D | Hot ACN/water | C | 5 | 343 | ND | 67 | ND |
| | slurry | | | | | | |
| E | ACN/water soaking | | 4 | 51 | ND | 9 | 19 |
| F | Hot ACN slurry | B | 5 | 508 | ND | 97 | 31 |
| G | ACN soaking | | 2 | 110 | ND | 147 | ND |

Processes involving acetonitrile were effective at achieving ICH levels for the residual solvents and presented good operativity because acetonitrile increased the wettability of thiostrepton. The soaking processes were easier to operate and minimized mechanical losses as compared with hot slurry processes. The ACN/water soaking resulted in lower methanol and ACN levels than ACN-only soaking.

To finalize the development and define the strategy for the residual solvent displacement process, the resulting product from the scaled-up crystallization in chloroform and acetonitrile (Batch H) was divided into 3 parts and the soaking process was reproduced with ACN only, a mixture of water/ACN 8/2 and water only. The soaking process that was effective to achieve ICH levels for the residual solvents proved to be water/ACN 8/2 (Batch I; see table below). In none of the experiments was a change in the impurity profile nor degradation observed.

| | | | | | | | Water | | |
|---|---|---|---|---|---|---|---|---|---|
| | | Scale | Residual Solvents (ppm) | | | | Content | Purity | Yield |
| Batch | Conditions | (g) | MeOH | DCM | CHCl₃ | ACN | (%) | (%) | (%) |
| ICH limits | — | — | 3000 | 600 | 60 | 410 | — | >98.0 | — |
| Commercial | Starting | — | 39572 | 54 | 2291 | N/A | 2.27 | 97.7 | — |
| TS | Material | | | | | | | | |
| H | Crystalization | 15 | 82 | ND | 7020 | 30733 | 1.37 | 98.4 | 85 |
| | CHCl₃/ACN | | | | | | | | |
| I | Water/ACN | 4 | 270 | ND | 27 | 55 | 1.93 | 98.5 | Estimated* |
| | 8/2 soaking | | | | | | | | 90 |
| J | ACN soaking | 3 | 88 | ND | 259 | 444 | 2.37 | 98.6 | 92 |
| K | Water | 4 | 113 | ND | 3578 | 51 | 1.92 | 98.7 | 93 |
| | soaking | | | | | | | | |

### Example 5: Comparison of Biological Activity of Ultrapure Thiostrepton and Commercial Thiostrepton

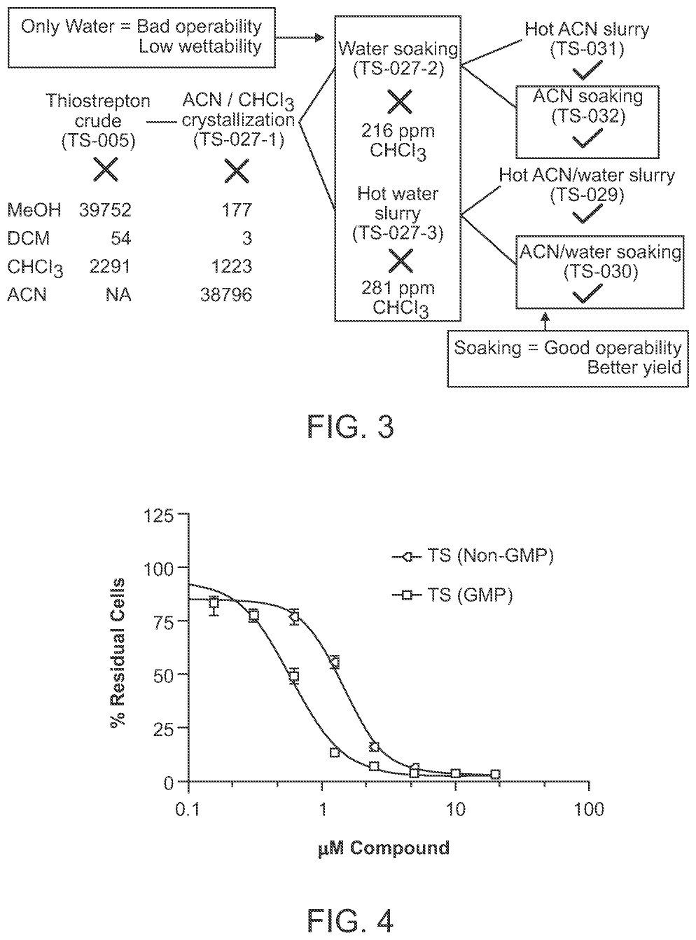

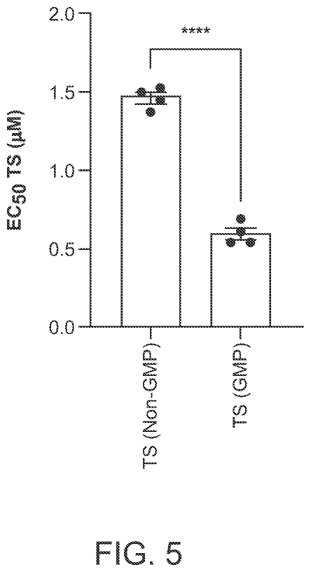

Commercial thiostrepton (TS) starting material (TS Non-GMP) and ultrapure TS (TS GMP) were diluted to 10 mM in sterile DMSO. Lots of TS were evaluated in a cell viability assay using the ovarian cancer cell line SKOV3 purchased from ATCC. Two replicate plates containing technical duplicates were set up for screening. Cells were plated at a density of 2,500 cells/well in 96-well plates and allowed to adhere for 24 hours prior to treatment. A starting concentration of 20 μM for each TS solution was serial diluted 1:1 covering a concentration range of 20 μM to 20 nM. Cells were incubated with treatments for 48 hours prior to fixation with 3% formaldehyde and crystal violet cell staining to determine residual cells remaining (100%=no cell death, 0%=complete cell death). FIG. 4. EC50 values (the concentration required to kill 50% of cells) were determined using a variable slope least square fit non-linear regression. FIG. 5. The data shown that ultrapure TS exhibits better biological activity than commercial TS both as measured by % cell death of cancer cells and EC50.

### Example 6: Exemplary Pharmaceutical Composition Comprising Ultrapure Thiostrepton

| | Quantity | | Quantity | |
|---|---|---|---|---|
| Composition per vial | per vial | | per batch | |
| Ultrapure Thiostrepton* | 90.0 | mg | 390.0 | g |
| Dimethyl sulfoxide (DMSO) | 528.8 | mg | 2291.3 | g |
| Vitamin E - TPGS | 2.1 | g | 9100.0 | g |
| Tris(hydroxymethyl)aminomethane | 18.17 | mg | 78.74 | g |

| | | |
|---|---|---|
| Hydrochloric acid 1N | Q.S. pH 7.4 | Q.S. pH 7.4 |
| Water for injection | Q.S. 30.3 g | Q.S. 131.2 Kg |
| | (30.0 mL) | (130 L) |

*Component adjusted according to its wet substance potency.

### INCORPORATION BY REFERENCE

All of the U.S. patents, and U.S. and PCT published patent applications cited herein are hereby incorporated by reference.

### EQUIVALENTS

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

1. An ultrapure preparation of thiostrepton having a purity of at least about 98% (w/w); wherein the preparation comprises less than or equal to:
a. 3000 ppm methanol, preferably less than or equal to 300 ppm methanol;
b. 600 ppm dichloromethane, preferably less than or equal to 60 ppm dichloromethane;
c. 60 ppm chloroform; and
d. 410 ppm acetonitrile, preferably less than or equal to 200 ppm acetonitrile.

2. The ultrapure preparation of thiostrepton of claim 1, wherein the purity of thiostrepton is at least about 99% (w/w).

3. A method of purifying thiostrepton comprising the steps of:
(1) dissolving thiostrepton in a first solvent to generate a first thiostrepton solution;
(2) distilling solvent impurities from the first thiostrepton solution to generate a second thiostrepton solution;
(3) combining a second solvent with the second thiostrepton solution to precipitate thiostrepton and thus generate a first thiostrepton solid and a third solution;
(4) washing the first thiostrepton solid with a third solvent to remove impurities and thus generate a second thiostrepton solid; and
(5) drying the second thiostrepton solid to remove residual solvent;
thereby producing an ultrapure preparation of thiostrepton.

4. The method of claim 3, wherein the first solvent comprises chloroform, and optionally from 0.5 to 1.0% (v/v) ethanol.

5. The method of claim 3, wherein:
step (1) further comprises cooling the first thiostrepton solution to a temperature of from 15 to 30° C.; and/or
step (2) reduces the volume of the second thiostrepton solution to at least 50% less than the volume of the first thiostrepton solution and optionally comprises cooling the second thiostrepton solution to a temperature of from 15° C. to 25° C.; and/or
the second solvent in step (3) comprises acetonitrile, preferably at a volume equal to the volume of the second thiostrepton solution and wherein step (3) optionally further comprises separating the first thiostrepton solid from the third solution and drying the first thiostrepton solid; and/or
the third solvent in step (4) comprises water, or is a mixture of water and acetonitrile, preferably a 4:1 v/v mixture of water and acetonitrile, and wherein the washing of the first thiostrepton solid with a third solvent of step (4) optionally comprises soaking the first thiostrepton solid with a third solvent.

6. The method of claim 3, wherein the method comprises the additional step of washing the second thiostrepton solid with at least one further portion of the third solvent following the step of washing the first solid with a third solvent to generate the second solid.

7. The method of claim 3, wherein the method comprises the additional step of washing the second thiostrepton solid with a fourth solvent, preferably soaking the second thiostrepton solid with the fourth solvent, following the step of washing the first thiostrepton solid with a third solvent to generate the second thiostrepton solid, and wherein said fourth solvent optionally comprises water.

8. The method of claim 3, wherein the second thiostrepton solid is analyzed for methanol, acetonitrile, dichloromethane and/or chloroform content and wherein the second thiostrepton solid is subjected to further cycles of steps (4) and (5) if the second thiostrepton solid comprises greater than 3000 ppm methanol, 600 ppm dichloromethane, 60 ppm chloroform, and/or 410 ppm acetonitrile.

9. The method of claim 3, wherein step (1) is performed on a thiostrepton scale of from 100 milligrams to 100 kilograms, preferably from 1.0 gram to 10 kilograms.

10. An ultrapure preparation of thiostrepton prepared according to the method of claim 3.

11. A pharmaceutical composition, comprising the ultrapure preparation of thiostrepton of claim 1; and one or more pharmaceutically acceptable excipients or carriers.

12. The pharmaceutical composition of claim 11, wherein the pharmaceutical composition is an aqueous composition, preferably comprising from 1 to 5 mg of ultrapure thiostrepton per mL of water.

13. The pharmaceutical composition of claim 11, further comprising Vitamin E-TPGS, preferably at an amount of from 0.05 to 0.1 g of Vitamin E-TPGS per mL of water.

14. The pharmaceutical composition of claim 11, further comprising dimethyl sulfoxide (DMSO), preferably at an amount of from 0.01 to 0.03 g of DMSO per mL of water.

15. A method of treating cancer comprising administering to a subject in need thereof a pharmaceutical composition according to claim 11.

16. The method of claim 5, wherein the method comprises the additional step of washing the second thiostrepton solid with at least one further portion of the third solvent following the step of washing the first solid with a third solvent to generate the second solid.

17. The method of claim 16, wherein the method comprises the additional step of washing the second thiostrepton solid with a fourth solvent, preferably soaking the second thiostrepton solid with the fourth solvent, following the step of washing the first thiostrepton solid with a third solvent to generate the second thiostrepton solid, and wherein said fourth solvent optionally comprises water.

18. The method of claim 17, wherein the second thiostrepton solid is analyzed for methanol, acetonitrile, dichloromethane and/or chloroform content and wherein the second thiostrepton solid is subjected to further cycles of steps (4) and (5) if the second thiostrepton solid comprises greater than 3000 ppm methanol, 600 ppm dichloromethane, 60 ppm chloroform, and/or 410 ppm acetonitrile.
